(19) United States
(12) Patent Application Publication
(10) Pub. No.: US 2025/0057157 A1
(43) Pub. Date: Feb. 20, 2025

(54) Universal Formulation

(71) Applicant: Gama Healthcare Limited, Hemel Hempstead, Hertfordshire (GB)

(72) Inventor: James Giles Crowley, Yorkshire (GB)

(21) Appl. No.: 18/720,236

(22) PCT Filed: Dec. 16, 2022
(86) PCT No.: PCT/GB2022/053279

§ 371 Date: Jun. 14, 2024

(30) Foreign Application Priority Data
Dec. 17, 2021 (GB) 2118392.6

(51) Int. Cl. A01N 33/24; A01N 25/34; A01N 31/02; A01N 37/44; A01N 43/36; A01P 1/00

(52) U.S. Cl. CPC A01N 33/24; A01N 25/34; A01N 31/02; A01N 37/44; A01N 43/36; A01P 1/00

(57) ABSTRACT

The invention is directed to a disinfectant composition.

The present invention relates to a composition comprising at least one quaternary ammonium compound, at least one alcohol ethoxylate and at least one amine oxide, wherein the at least one quaternary ammonium compound comprises at least one polyquaternium compound, a wet wipe comprising a substrate that has been impregnated with said composition as well as the use of said composition or of said wet wipe for disinfection of a surface.

In a healthcare environment, there are a very wide range of surfaces, equipment, and devices which must not only be kept clean, but also place special demands on the cleaning products used. In particular, it must be taken into account that in the medical industry the surfaces in question must be hygienic on the one hand and that the cleaning products must not fatigue the materials too much on the other hand. Typically, such items are made of stainless steel, other metals, glass and/or a wide range of plastics and rubbers. They may include screens, keyboards, trays, wheelchairs, trolleys, walls, windows and many diverse pieces of medical equipment and equipment stands, bed frames, mattresses, commodes and furniture.

As the infection control market has evolved the requirement of broad-spectrum products, which would include efficacy against more resilient microorganisms such as non-enveloped viruses. These organisms are highly infectious pathogens responsible for significant healthcare associated infections. Non-enveloped viruses are a challenge for disinfection due to the structural differences of the capsid core, availability and number of targets, and accessibility to the nucleic acid.

Wet wipes comprising a substrate that has been impregnated with a disinfecting composition have been on the market for years and have provided the healthcare services and other areas with a simple, effective infection control solution.

Whilst said wipes possess broad antimicrobial capability, the performance in short contact times against adenovirus and norovirus could be improved and good material compatibility, especially with polymer surfaces, would be desirable.

Further, the results obtained against yeast, in particular Candida albicans and non-enveloped viruses (in particular noro and adeno) could be improved.

EP1634942 A1 relates to an acid disinfectant and cleaning composition containing one or more selected quaternary ammonium salts, one or more selected alcohol alkoxylates, one or more alcohols and one or more hydroxycarboxylic acids.

WO2019051310 A1 relates to an alcohol-potentiated antimicrobial soap formulation containing an alcohol-potentiated antimicrobial agent.

However, previous formulations for disinfecting hands or plastic surfaces use alcohol-based solvents; moreover, these formulations have an acidic pH or contain high concentrations of active ingredients. In particular, the high alcohol content of these disinfectants causes them to evaporate quickly on the surface, so they have a strong drying effect on the hands and a short exposure time is required to effectively kill viruses or bacteria, for example. The short exposure times of such disinfectant formulations with high (+20%) alcohol content in turn require high concentrations of active ingredients (biocides) and an acidic or basic pH. Alcohol-containing formulations also pose an additional risk during production, as the ingredients are flammable.

The formulations known from the prior art are therefore of limited suitability for use in hand or surface disinfection, as they are harmful to the skin and have the potential to damage polymer surfaces.

In particular, the negative effects of prior art disinfectants on human skin, must be taken into account. Due to a non-neutral pH value, high concentration of biocides and a high alcoholic content in the overall composition, hands are quickly dried out and thus become more sensitive to external influences.

Similar effects can also be observed on plastic surfaces, as plasticizers, for example, are more easily dissolved from the material and thus the surface becomes brittle.

It is an object of the present invention to provide a composition that is efficacious in short, appropriate and relevant contact times for healthcare settings, in particular, that has improved activity against viruses in relevant short contact times that are required in hospital disinfection. Another objective is to achieve the required performance of a number of known biocides, whilst maintaining surface and skin compatibility.

This objective was solved by a composition comprising at least one quaternary ammonium compound, at least one alcohol ethoxylate, at least one chelating agent, and at least one amine oxide, wherein the at least one quaternary ammonium compound comprises at least one polyquaternium compound.

The composition is not classified as harmful and does not contain any ingredients known or suspected to be harmful to humans. Further, the compositions according to the present invention is free of phenolic compounds and polyhexamethylene biguanide (PHMB).

Further, the compositions according to the present invention have a reduced content of alcohol ethoxylate nonionic surfactants when compared to previously described disinfectant compositions to optimize the biocide performance of the composition while improving material compatibility with polymer surfaces.

It has been surprisingly found that the composition according to the invention exhibits good antimicrobial action in suspension test in a clinically relevant time, although no biocide was added. It was also observed that the composition is capable of enhancing the antimicrobial activity of biocides.

Furthermore, it has been surprisingly found, that polyquaternium compounds achieve excellent results when used in a disinfectant composition. It is believed that the inclusion of a polyquaternium compound, in particular polyquaternium-6 (poly(dialyl)dimethylammoniumchloride), polyquaternium-4 (Hydroxyethyl cellulose dimethyl diallylammonium chloride copolymer), polyquaternium-5 (Copolymer of acrylamide and quaternized dimethylammoniumethyl methacrylate), polyquaternium-7 (Copolymer of acrylamide and diallyldimethylammonium chloride), polyquaternium-10 (Quaternized hydroxyethyl cellulose), polyquaternium-11 (Copolymer of vinylpyrrolidone and quaternized dimethylaminoethyl methacrylate), polyquaternium-14 (Trimethylaminoethylmethacrylate homopolymer), polyquaternium-16 (Copolymer of vinylpyrrolidone and quaternized vinylimidazole), polyquaternium-22 (Copolymer of acrylic acid and diallyldimethylammonium Chloride), polyquaternium-28 (Copolymer of vinylpyrrolidone and methacrylamidopropyl trimethylammonium), polyquaternium-29 (Chitosan modified with propylene oxide and quaternized with epichlorhydrin), polyquaternium-39 (Terpolymer of acrylic acid, acrylamide and diallyldimethylammonium Chloride), polyquaternium-46 (Terpolymer of vinylcaprolactam, vinylpyrrolidone, and quaternized vinylimidazole), or any mixtures thereof, in combination with the other constituents of the composition, gives raise to the excellent properties of the composition, in particular concerning the high efficiency against Candida albicans and non-enveloped viruses like noro virus or adeno virus.

In one preferred embodiment, the polyquaternium compound is polyquaternium-6.

The composition provides a range of antimicrobial activity against bacteria, yeast, enveloped and non-enveloped viruses (like noro and adeno virus).

Particular advantages of the compositions according to the present invention are high efficiency against bacteria, yeasts, enveloped and non-enveloped viruses while at the same time showing low inherent toxicity and are not harsh to hands and plastic materials and therefore avoid damage to both skin and plastic (environmental stress tracking).

Polyquaternium is the International Nomenclature for Cosmetic Ingredients designation for several polycationic polymers. Polyquaternium is a neologism used to emphasize the presence of quaternary ammonium centers in the polymer. Different polymers are distinguished by the numerical value that follows the word “polyquaternium”. Polyquaternium-5, polyquaternium-7, and polyquaternium-47 are three examples, each a chemically different type of polymer. The numbers are assigned in the order in which they are registered rather than because of their chemical structure.

The following table lists the identity of each of the polyquaternium compounds:

| Polyquaternium | Chemical Identity |
|---|---|
| Polyquaternium-1 | Ethanol, 2,2′,2″ -nitrilotris-, polymer with 1,4-dichloro-2- |
| | butene and N,N,N′,N′-tetramethyl-2-butene-1,4-diamine |
| Polyquaternium-2 | Poly[bis(2-chloroethyl) ether-alt-1,3-bis[3- |
| | (dimethylamino)propyl]urea] |
| Polyquaternium-4 | Hydroxyethyl cellulose dimethyl diallylammonium chloride |
| | copolymer; Diallyldimethylammonium chloride-hydroxyethyl |
| | cellulose copolymer |
| Polyquaternium-5 | Copolymer of acrylamide and quaternized |
| | dimethylammoniumethyl methacrylate |
| Polyquaternium-6 | Poly(diallyldimethylammonium chloride) |
| Polyquaternium-7 | Copolymer of acrylamide and diallyldimethylammonium |
| | chloride |
| Polyquaternium-8 | Copolymer of methyl and stearyl dimethylaminoethyl ester of |
| | methacrylic acid, quaternized with dimethylsulphate[2] |
| Polyquaternium-9 | Homopolymer of N,N-(dimethylamino)ethyl ester of |
| | methacrylic acid, quaternized with bromomethane |
| Polyquaternium-10 | Quaternized hydroxyethyl cellulose |
| Polyquaternium-11 | Copolymer of vinylpyrrolidone and quaternized |
| | dimethylaminoethyl methacrylate |
| Polyquaternium-12 | Ethyl methacrylate / abietyl methacrylate / diethylaminoethyl |
| | methacrylate copolymer quaternized with dimethyl sulfate |
| Polyquaternium-13 | Ethyl methacrylate / oleyl methacrylate / diethylaminoethyl |
| | methacrylate copolymer quaternized with dimethyl sulfate |
| Polyquaternium-14 | Trimethylaminoethylmethacrylate homopolymer |
| Polyquaternium-15 | Acrylamide-dimethylaminoethyl methacrylate methyl chloride |
| | copolymer |
| Polyquaternium-16 | Copolymer of vinylpyrrolidone and quaternized vinylimidazole |
| Polyquaternium-17 | Adipic acid, dimethylaminopropylamine and dichloroethylether |
| | copolymer |
| Polyquaternium-18 | Azelaic acid, dimethylaminopropylamine and |
| | dichloroethylether copolymer |
| Polyquaternium-19 | Copolymer of polyvinyl alcohol and 2,3-epoxypropylamine |
| Polyquaternium-20 | Copolymer of polyvinyl octadecyl ether and 2,3- |
| | epoxypropylamine |
| Polyquaternium-22 | Copolymer of acrylic acid and diallyldimethylammonium |
| | Chloride |
| Polyquaternium-24 | Quaternary ammonium salt of hydroxyethyl cellulose reacted |
| | with a lauryl dimethyl ammonium substituted epoxide. |
| Polyquaternium-27 | Block copolymer of Polyquaternium-2 and Polyquaternium-17 |
| Polyquaternium-28 | Copolymer of vinylpyrrolidone and methacrylamidopropyl |
| | trimethylammonium |
| Polyquaternium-29 | Chitosan modified with propylen oxide and quaternized with |
| | epichlorhydrin |
| Polyquaternium-30 | Ethanaminium, N-(carboxymethyl)-N,N-dimethyl-2-[(2-methyl- |
| | 1-oxo-2-propen-1-yl)oxy]-, inner salt, polymer with methyl 2- |
| | methyl-2-propenoate |
| Polyquaternium-31 | N,N- dimethylaminopropyl-N-acrylamidine quatemized with |
| | diethylsulfate bound to a block of polyacrylonitrile |
| Polyquaternium-32 | Poly(acrylamide 2-methacryloxyethyltrimethyl ammonium |
| | chloride) |
| Polyquaternium-33 | Copolymer of trimethylaminoethylacrylate salt and acrylamide |
| Polyquaternium-34 | Copolymer of 1,3-dibromopropane and N,N-diethyl-N′,N′- |
| | dimethyl-1,3-propanediamine |
| Polyquaternium-35 | Methosulphate of the copolymer of |
| | methacryloyloxyethyltrimethylammonium and of |
| | methacryloyloxyethyldimethylacetylammonium |
| Polyquaternium-36 | Copolymer of N,N-dimethylaminoethylmethacrylate and |
| | buthylmethacrylate, quaternized with dimethylsulphate |
| Polyquaternium-37 | Poly(2-methacryloxyethyltrimethylammonium chloride) |
| Polyquaternium-39 | Terpolymer of acrylic acid, acrylamide and |
| | diallyldimethylammonium Chloride |
| Polyquaternium-42 | Poly[oxyethylene(dimethylimino)ethylene |
| | (dimethylimino)ethylene dichloride] |
| Polyquaternium-43 | Copolymer of acrylamide, acrylamidopropyltrimonium |
| | chloride, 2-amidopropylacrylamide sulfonate and |
| | dimethylaminopropylamine |
| Polyquaternium-44 | 3-Methyl-1-vinylimidazolium methyl sulfate-N-vinylpyrrolidone |
| | copolymer |
| Polyquaternium-45 | Copolymer of (N-methyl-N-ethoxyglycine) methacrylate and |
| | N,N-dimethylaminoethylmethacrylate, quaternized with |
| | dimethyl sulphate |
| Polyquaternium-46 | Terpolymer of vinylcaprolactam, vinylpyrrolidone, and |
| | quaternized vinylimidazole |
| Polyquaternium-47 | Terpolymer of acrylic acid, methacrylamidopropyl |
| | trimethylammonium chloride, and methyl acrylate |

In connection with the definition of polyquaternium compounds as used in this application, we make further reference to the following patent literature:
Iwata, Hiroshi; Shimada, Kunio (2012-10-02). Formulas, Ingredients and Production of Cosmetics: Technology of Skin-and Hair-Care Products in Japan. Springer Science & Business Media. ISBN 9784431540618.
Schueller, Randy; Romanowski, Perry (1999-02-02). Conditioning Agents for Hair and Skin. CRC Press. ISBN 9780824719210.

In a preferred embodiment, the at least one polyquaternium compound is selected from polyquaternium-1, polyquaternium-2, polyquaternium-4, polyquaternium-5, polyquaternium-6, polyquaternium-7, polyquaternium-8, polyquaternium-9, polyquaternium-10, polyquaternium-11, polyquaternium-12, polyquaternium-13, polyquaternium-14, polyquaternium-15, polyquaternium-16, polyquaternium-17, polyquaternium-18, polyquaternium-19, polyquaternium-20, polyquaternium-22, polyquaternium-24, polyquaternium-27, polyquaternium-28, polyquaternium-29, polyquaternium-30, polyquaternium-31, polyquaternium-32, polyquaternium-33, polyquaternium-34, polyquaternium-35, polyquaternium-36, polyquaternium-37, polyquaternium-39, polyquaternium-42, polyquaternium-43, polyquaternium-44, polyquaternium-45, polyquaternium-46, polyquaternium-47, and any mixtures thereof.

In a preferred embodiment, the at least one polyquaternium compound is polyquaternium-6 (poly(dialyl)dimethylammoniumchloride), polyquaternium-4 (Hydroxyethyl cellulose dimethyl diallylammonium chloride copolymer), polyquaternium-5 (Copolymer of acrylamide and quaternized dimethylammoniumethyl methacrylate), polyquaternium-7 (Copolymer of acrylamide and diallyldimethylammonium chloride), polyquaternium-10 (Quaternized hydroxyethyl cellulose), polyquaternium-11 (Copolymer of vinylpyrrolidone and quaternized dimethylaminoethyl methacrylate), polyquaternium-14 (Trimethylaminoethylmethacrylate homopolymer), polyquaternium-16 (Copolymer of vinylpyrrolidone and quaternized vinylimidazole), polyquaternium-22 (Copolymer of acrylic acid and diallyldimethylammonium Chloride), polyquaternium-28 (Copolymer of vinylpyrrolidone and methacrylamidopropyl trimethylammonium), polyquaternium-29 (Chitosan modified with propylene oxide and quaternized with epichlorhydrin), polyquaternium-39 (Terpolymer of acrylic acid, acrylamide and diallyldimethylammonium Chloride), polyquaternium-46 (Terpolymer of vinylcaprolactam, vinylpyrrolidone, and quaternized vinylimidazole), or any mixtures thereof.

The most preferred polyquaternium compound is polyquaternium-6.

While the selection of the at least one alcohol ethoxylate is not generally limited, preferably the at least one alcohol ethoxylate comprises alcohol ethoxylate nonionic with HLB from 4-16.

In a preferred embodiment, the at least one alcohol ethoxylate comprises alcohol ethoxylate nonionic with HLB from 8-14, more preferably 11-13.

In a preferred embodiment, the at least one alcohol ethoxylate comprises alcohol ethoxylate nonionic C9-11, EO6 (C9-11 pareth 6).

Usually, alcohol ethoxylates have multifunctional properties, which include detergency, foaming, builders and lowering surface tension. The addition of the alcohol ethoxylate(s) improves the solubilization of fats and proteins, which may aid microbiological activity.

Alcohol ethoxylates are a group of nonionic surfactants that are obtained by alkoxylation, i.e. by reacting ethylene oxide, propylene oxide or butylene oxide (preferably ethylene oxide) with primary long-chain fatty-or oxo-alcohols in the presence of basic or acidic catalysts at temperatures of 120-200° C. and pressures of 1-10 bar.

In one embodiment, the composition according to the present invention comprises a primary alcohol ethoxylate in an amount of 0.01 to 0.5% w/w.

In one embodiment, the composition according to the present invention comprises at least one amine oxide compound in an amount of 0.01 to 1% w/w.

In one embodiment, the composition according to the present invention comprises N,N-dimethyldecylamine-N-oxide as the at least one amine oxide compound.

In one embodiment, the composition according to the present invention comprises at least one chelating agent in an amount of 0.01 to 1% w/w.

In one embodiment, the composition according to the present invention comprises Disodium ethylenediaminetetraacetic acid as the at least one chelating agent.

In one embodiment, the composition according to the present invention comprises at least one polyquaternium compound in an amount of 0.01 to 1.0% w/w.

In one embodiment, the composition comprises at least one preservative. A preservative is a substance or chemical that is added to prevent decomposition by microbial growth or by undesirable chemical changes. The preservative is preferably an antimicrobial preservative that prevents degradation by bacteria.

In one embodiment, the composition according to the present invention comprises 2-phenoxy ethanol as preservative.

In one embodiment, the composition according to the present invention is free of 2-phenoxy ethanol.

In one embodiment, the composition according to the present invention additionally comprises benzalkonium chloride and/or didecyldimethylammonium chloride as additional quaternary ammonium compounds.

In one embodiment, the composition according to the present invention comprises a further quaternary ammonium compound in form of C12-C16 Benzalkonium Chloride, in particular in an amount of 0.01 to 0.7% w/w.

In one embodiment, the composition according to the present invention comprises a further quaternary ammonium compound in form of Didecylydimethyl ammonium chloride, in particular in an amount of 0.01 to 0.7% w/w.

In one embodiment, the composition according to the present invention comprises at least one preservative in an amount of 0.01 to 1% w/w.

In one embodiment, the composition according to the present invention further comprises one or more additives selected from a group consisting of one or more disinfectants, stabilizers, preservatives, dyes, fragrances, odor masking agents and/or mixtures thereof.

In one embodiment, the composition according to the present invention is in form of an aqueous solution or dispersion.

In a preferred embodiment, the composition according to the invention is in the form of an aqueous solution having a pH of from 3 to 9, preferably from 4 to 8, more preferably from 5 to 7.

The composition according to the present invention is preferably characterised in that the composition comprises
from 0.01 to 0.25% w/w N,N-dimethyldecylamine-N-oxide,
from 0.01 to 0.1% w/w Alcohol exthoxylate nonionic C9-11, E06,
from 0.01 to 1.0% w/w Poly(dialyl dimethylammoniumm Chloride),
from 0.01 to 1.0% w/w Disodium ethylenediaminetetraacetic acid,
wherein the solvent comprises water, preferably the solvent is pure water.

In a further embodiment, the composition according to the present invention is characterised in that the composition comprises
from 0.01 to 0.7% w/w C12-C16 Benzalkonium Chloride,
from 0.01 to 0.7% w/w Didecylydimethyl ammonium chloride,
from 0.01 to 0.25% w/w N,N-dimethyldecylamine-N-oxide,
from 0.01 to 0.1% w/w Alcohol exthoxylate nonionic C9-11, E06,
from 0.01 to 1.0% w/w Poly(dialyl dimethylammoniumm Chloride),
from 0.01 to 1.0% w/w Disodium ethylenediaminetetraacetic acid,
with the solvent comprises water, preferably the solvent is pure water.

In a further embodiment, the composition according to the present invention is characterised in that the composition comprises
from 0.01 to 1.0% w/w phenoxyethanol, such as 0.01 to 1.0% w/w,
from 0.01 to 0.7% w/w C12-C16 Benzalkonium Chloride,
from 0.01 to 0.7% w/w Didecylydimethyl ammonium chloride,
from 0.01 to 0.25% w/w N,N-dimethyldecylamine-N-oxide,
from 0.01 to 0.1% w/w Alcohol exthoxylate nonionic C9-11, E06,
from 0.01 to 1.0% w/w Poly(dialyl dimethylammoniumm Chloride),
from 0.01 to 1.0% w/w Disodium ethylenediaminetetraacetic acid,
with the solvent comprises water, preferably the solvent is pure water.

The present invention further concerns a wet wipe comprising a substrate that has been impregnated with a composition as described in the preceding sections.

In a further embodiment, the composition according to the invention is applied to a surface by means of a sprayer, dispenser or comparable device.

The substrate that is impregnated with the composition is preferably a nonwoven material. Suitable nonwoven materials include but are not limited to those types which are binder free so that the binder is not deleteriously affected by the composition nor itself contributes to smearing. Examples of binder free nonwoven materials include spun laced or hydro-entangled nonwoven materials. However other types such as wet laid, airlaid, thermobond or stitch bonded types may also be used.

The wipes may comprise fibres made of any of or a mixture of polypropylene, polyester, polyethylene, viscose, cotton, regenerated wood pulp and cellulose. They may also include micro-fibre and nano-fibre products.

The substrate is preferably produced in the form of individual tissues or a perforated roll of material from which individual tissues can be separated that are impregnated with the composition and packaged ready to be dispensed from resealable tubs, buckets, flow-wrap packs or similar. Alternatively, impregnated wipes may be individually sealed in a wrapper made of a suitable packaging material, such as an impermeable foil, cellophane and the like.

The ingredients of the inventive compositions are simply mixed together to form an aqueous or aqueous alcohol solution or dispersion that can then be used to impregnate a substrate by soaking the substrate in the composition to thereby produce a wet wipe in accordance with the invention.

In an alternative embodiment, the composition according to the invention is not provided as an aqueous or alcoholic solution, but as a concentrate, which can be diluted accordingly by adding a suitable solvent.

Such a wet wipe is suitable for cleaning a wide range of surfaces and materials and removing various types and levels of soiling, both organic and inorganic in a manner that leaves a clean and disinfected surface.

The present invention further concerns the use of a composition or of a wet wipe as described in the preceding for disinfection of a surface.

The present invention is further illustrated by the following non-limiting examples.

### EXAMPLES

The following composition were prepared by mixing the listed ingredients in water.

### Part A

### Examples

Possible preferred inclusion ranges for the individual components are given in table 1. When a lower range of 0 is given, this can mean that the component is not contained in the composition or that this component is contained in the composition in an amount of more than 0, hence 0 is excluded from the given range.

TABLE 1

| | | | | Potential |
|---|---|---|---|---|
| | | | | Inclusion |
| | | | CAS | Range |
| Ingredient | Function | INCI Name | Number | (% w/w) |
| N,N- | Surfactant | Decylamine oxide | 2605-79-0 | 0.01-0.25 |
| dimethyldecylamine -N- | | | | |
| oxide | | | | |
| Alcohol Exthoxylate | Surfactant | C9-11 Pareth 6 | 68439-46-3 | 0.01-1 |
| nonionic C9-11, EO6 | | | | |
| Poly(diallyl | Skin / Wipe | Polyquaternium 6 | 26062-79-3 | 0.01-1 |
| dimethylammonium | conditioning | | | |
| Chloride) | agent, | | | |
| | performance | | | |
| | booster | | | |
| Disodium | Chelating | Disodium EDTA | 139-33-3 | 0.01-1 |
| ethylenediaminetetra | | | | |
| acetic acid | | | | |
| Aqua | Solvent | Aqua | 7732-18-5 | To 100% |

A preferred composition according to the invention is given in the following table 2 and is referred to as inventive example (IE1) in the following:

TABLE 2

| | | | | Inclusion |
|---|---|---|---|---|
| | | | CAS | Level |
| Ingredient | Function | INCI Name | Number | (% w/w) |
| N,N- | Surfactant | Decylamine oxide | 2605-79-0 | 0.15 |
| dimethyldecylamine -N- | | | | |
| Alcohol Exthoxylate | Surfactant | C9-11 Pareth 6 | 68439-46-3 | 0.06 |
| nonionic C9-11, EO6 | | | | |
| Poly (diallyl | Skin / Wipe | Polyquaternium 6 | 26062-79-3 | 0.05 |
| dimethylammonium | conditioning | | | |
| Chloride) | agent, | | | |
| | performance | | | |
| | booster | | | |
| Disodium | Chelating | Disodium EDTA | 139-33-3 | 0.03 |
| ethylenediaminetetra | | | | |
| acetic acid | | | | |
| Aqua | Solvent | Aqua | 7732-18-5 | To 100% |

The inventive composition according to table 2 (IE1) has been tested to measure the improvements of microbiological efficacy (against Staphylococcus aureus, Pseudomonas aeruginosa, Enterococcus hirae, Escherichia coli, and Candida albicans) of the inventive composition and improvement of broad-spectrum efficacy following standard methods. The microbiologically efficacy was tested following the EN standards for yeast/fungi (EN 13624) or bacteria (EN 13727). All testing carried out using clinically relevant, medical dirty conditions, to ensure the formulation/wipes work under the most stringent, clinically relevant condition.

TABLE 3
Inventive composition according to table 2.

| | | [log | |
|---|---|---|---|
| | | reduction]: | |
| | | Contact | |
| EN Test | Organism | time <= 2 min | Conclusion |
| 13727 | S. aureus | ≥3 | Co-formulant mixture has |
| | | | some antimicrobial activity |
| 13727 | P. aeruginosa | ≥3 | Co-formulant mixture has |
| | | | some antimicrobial activity |
| 13727 | E. hirae | ≥3 | Co-formulant mixture has |
| | | | some antimicrobial activity |
| 13727 | E. coli | ≥3 | Co-formulant mixture has |
| | | | some antimicrobial activity |
| 13624 | C. Albicans | ≥3 | Co-formulant mixture has |
| | | | some antimicrobial activity |

The inventive composition according to table 2 (IE1) shows a log reduction of over 3 for all tested bacteria and yeast/fungi. From this it can be concluded that the composition according to the invention exhibits good antimicrobial action in suspension test in a clinically relevant time, although no biocide was added.

Further tests according to EN13727/EN13624 (organism specific) were conducted to verify the increased antimicrobial activity of the composition of the invention according to Table 2 (IE1) by the addition of a biocide (BZK=C12-C16

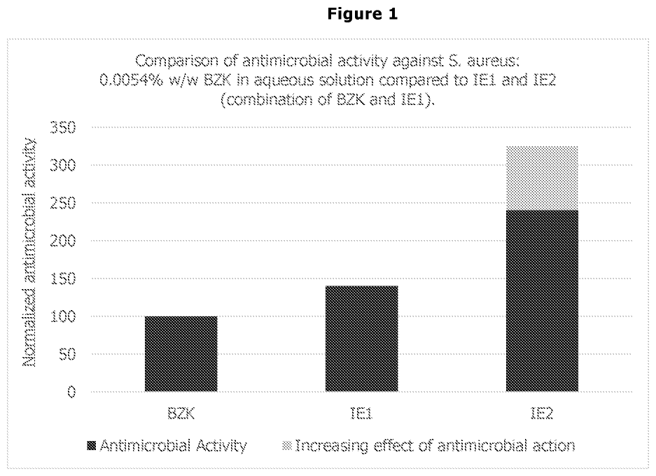

Benzalkonium Chloride) to give IE2. Data were generated under medically contaminated conditions with a contact time of 30 seconds. The antimicrobial activity of BZK alone is normalized as 100. The results of this test is shown in FIG. 1.

TABLE 4

| | | | | Potential |
|---|---|---|---|---|
| | | | | Inclusion |
| | | | CAS | Range (% |
| Ingredient | Function | INCI Name | Number | w/w) |
| 2-phenoxyethanol | Preservative/ | Phenoxyethanol | 122-99-6 | 0-1 |
| | Biocide | | | |
| C12-C16 Benzalkonium | Biocide | Benzalkonium | 68424-85-1 | 0-0.7 |
| Chloride | | Chloride | | |
| Didecylydimethyl | Biocide | Didecyldimonium | 7173-51-5 | 0-0.7 |
| ammonium chloride | | chloride | | |
| N,N- | Surfactant | Decylamine oxide | 2605-79-0 | 0.01-0.25 |
| dimethyldecylamine -N- | | | | |
| oxide | | | | |
| Alcohol Exthoxylate | Surfactant | C9-11 Pareth 6 | 68439-46-3 | 0.01-0.1 |
| nonionic C9-11, EO6 | | | | |
| Poly(diallyl | Skin / Wipe | Polyquaternium 6 | 26062-79-3 | 0.01-1 |
| dimethylammonium | conditioning | | | |
| Chloride) | agent, | | | |
| | performance | | | |
| | booster | | | |
| Disodium | Chelating | Disodium EDTA | 139-33-3 | 0.01-1 |
| ethylenediaminetetra | | | | |
| acetic acid | | | | |
| Allergen free Green tea | Fragrance | Parfum | Mixture | 0-1 |
| perfume | | | | |
| Aqua | Solvent | Aqua | 7732-18-5 | To 100% |

A preferred composition according to the invention is given in the following table and is referred to as inventive example 3 (IE3) in the following:

TABLE 5

| | | | | Inclusion |
|---|---|---|---|---|
| | | | CAS | Level (% |
| Ingredient | Function | INCI Name | Number | w/w) |
| 2-phenoxyethanol | Preservative/ | Phenoxyethanol | 122-99-6 | 0.3 |
| | Biocide | | | |
| C12-C16 Benzalkonium | Biocide | Benzalkonium | 68424-85-1 | 0.27 |
| Chloride | | Chloride | | |
| Didecylydimethyl | Biocide | Didecyldimonium | 7173-51-5 | 0.27 |
| ammonium chloride | | chloride | | |
| N,N- | Surfactant | Decylamine oxide | 2605-79-0 | 0.15 |
| dimethyldecylamine-N- | | | | |
| Alcohol Exthoxylate | Surfactant | C9-11 Pareth 6 | 68439-46-3 | 0.06 |
| nonionic C9-11, EO6 | | | | |
| Poly (diallyl | Skin / Wipe | Polyquaternium 6 | 26062-79-3 | 0.05 |
| dimethylammonium | conditioning | | | |
| Chloride) | agent, | | | |
| | performance | | | |
| | booster | | | |
| Disodium | Chelating | Disodium EDTA | 139-33-3 | 0.03 |
| ethylenediaminetetra | | | | |
| acetic acid | | | | |
| Allergen free Green tea | Fragrance | Parfum | Mixture | 0.0075 |
| perfume | | | | |
| Aqua | Solvent | Aqua | 7732-18-5 | To 100% |

The inventive composition according to table 5 (IE3) has been tested to measure the improvements of microbiological efficacy (against Candida albicans) and improvement of broad-spectrum efficacy following standard methods. The microbiologically efficacy was tested following the EN standards for yeast/fungi (EN 13624). All testing carried out using clinically relevant, medical dirty conditions, to ensure the formulation/wipes work under the most stringent, clinically relevant condition.

The product is contacted with the fungi and at the end of the contacting time, the fungicidal action is neutralized, and the numbers of surviving fungi and reduction is calculated. The product shall demonstrate at least a 4 decimal log (lg) reduction.

Therefore, IE3 has been applied to different substrates and compared to non-inventive comparative examples referred to as CE1, CE2 and CE3.

The results of the EN13624 test is given in the following table 6.

TABLE 6

| | | | 2- | 2- | | Synper- | Decyl- | | | |
|---|---|---|---|---|---|---|---|---|---|---|
| | | | Phenoxy | Phenyl- | | onic | amine | Polyquat- | | Propylene |
| | BZK | DDAC | ethanol | phenol | 91-6 | PEL64 | oxide | ernium 6 | Glycerol | Glycol |
| CE1 | 0.54 | 0.54 | 0.6 | 0.1 | 0.99 | 0.12 | 0 | 0 | 0 | 0.2 |
| CE2 | 0.54 | 0.54 | 0.6 | 0.1 | 0.99 | 0.12 | 0 | 0 | 1 | 0 |
| CE3 | 0.54 | 0.54 | 0.6 | 0.1 | 0.99 | 0.12 | 0 | 0 | 1 | 0 |
| IE3 | 0.27 | 0.27 | 0.3 | 0 | 0.06 | 0 | 0.15 | 0.05 | 0 | 0 |

| | | | | | Allergen | | EN 13624 |
|---|---|---|---|---|---|---|---|
| | | | | | Free | | Candida Albicans |
| | | Di- | | | Green | | [log reduction]: |
| | | sodium | Citric | Sodium | Tea | | Contact |
| | Allantoin | EDTA | acid | Citrate | Perfume | pH | time <= 2 min |
| CE1 | 0 | 0 | 0.01 | 0.1 | 0.05 | 5.8 | 1.01 |
| CE2 | 0.1 | 0 | 0.01 | 0.1 | 0.015 | 5.8 | 1.96 |
| CE3 | 0 | 0 | 0.01 | 0.1 | 0.015 | 5.8 | 1.54 |
| IE3 | 0 | 0.03 | 0 | 0 | 0.0075 | 5.8 | >4.26 |

CE1-CE3: Comparative Examples 1 to 3
IE3: Inventive Examples 3 according to Table 5
BZK: C12-C16 Benzalkonium Chloride
DDAC: Didecylydimethyl ammonium chloride
91-6: Alcohol Exthoxylate nonionic C9-11, EO6
Synperonic PEL64: high MW, low HLB, ethylene oxide/propylene oxide block copolymer
Decyl-amine oxide N,N-dimethyldecylamine-N-oxide
Polyquaternium 6: Poly (diallyl dimethylammonium Chloride)
The inventive example IE 3 show a log reduction for Candida Albicans of more than 4 and this passed the test according to EN 13624.

### Part B

### 1.0 Introduction

The resistance to environmental stress cracking (ESC) of a medium viscosity polycarbonate (PC) commonly used in medical devices, has been evaluated in the presence of two GAMA Healthcare formulations. The polymer was provided by Resinex Ltd in the form of ‘chips’, these were subsequently injected moulded by Smithers at their site in Shrewsbury, UK. PC is an amorphous polymers, making it susceptible to ESC, used polymers are as follows:
Polycarbonate (medium viscosity)
Polycarbonate (high viscosity)
Polycarbonate and ABS blend
ABS
PMMA

Surface wet wipes used for disinfection all contain ‘ESC agents’, the levels and severity of which differ between products.

### 2.0 Test Methodology

Tested in general accordance with BS EN ISO 22088-3 ‘Plastics—Determination of resistance to environmental stress cracking (ESC)—Part 3: Bent strip method’.

This test involves taking test specimens and bending them at a fixed tensile strain. This is done using a stainless steel ‘strain jig’, these are metals formed with a constant radius of curvature. Tensile strain can be determined by the following equation:

$${ε}_{x} = \frac{h}{2 r + h}$$

εₓ=tensile strain, h=specimen thickness, r=radius of strain jig

The tests were performed on strain jigs at 0.5% tensile strain, 0.5% strain is in the range at which devices should be expected to be designed. Test specimens are bent over the strain jig, parallel to the curvature. The specimens are then exposed by application or immersion of the chemical liquid environment for a chosen time duration.

The specimens are observed throughout the testing for any crazing or cracking indicating ESC is taking place. For specimens in which ESC is unobservable, at the end of the exposure period for testing, these are tested for tensile properties according to ISO 527-2. The tensile properties of plastics undergoing ESC will change, with the plastic weakening, this additional testing can prove evidence of ESC failure in the absence of crazing or cracking. These are compared against unstrained/unexposed ‘control’ specimens.

### I. Test Specimens

Injected moulded tensile ‘dog-bones’ moulded to dimensions according to test specimen type 1A found in ISO 527-2. Strain jigs are designed for nominal 4 mm specimen thickness, all test specimens therefore have this thickness.

Specimens are conditioned to 23±2° C./50±10% RH for 24 hours before testing.

### II. Fitting Specimens to Strain Jigs

Test specimens are aligned with the face containing ejector pins (resulting from injection moulding) facing upwards for consistency.

Specimens lie parallel to each other and parallel to the sides of the jig. A maximum of three specimens are fitted to each jig.

Clamps are tightened evenly and sufficiently so that the gauge length, at least the central 75 mm, of the specimen are fully in contact with the curvature of the strain jig. The clamps must not be excessively tightened to avoid undue stress.

### III. Exposure to Chemical Environment

Very large volumes of liquid would be required to fully immerse the specimens and it would not be feasible to do so. As a result, the specimens were exposed to the chemical environment through use of cotton wool. Small pieces of cotton wool were cut and immersed in liquid before being placed on the centre of the specimens (see appendix). Liquid was added to the wool until it was saturated at the start and end of each business day (9 am/5 pm) to keep the specimens in contact with the fluids.

All specimens were tested in an environmental chamber set to 23° C./50% RH.

### IV. Observation & Evaluation

Test specimens were observed for signs of cracking three times per business day during the week of exposure. Following the week of testing the specimens were observed under low magnification with illumination to look for signs of crazing. Cracking sooner into the week indicates the chemical environment contains more aggressive ESC agents which are less compatible with the plastic tested.

The specimens that did not crack were tested for the tensile properties to look for changes in yield and break stress and strain. Samples were deemed to have failed if the mean break or yield stress were <80%, or mean break or yield strain were <50% of the unstrained/unexposed control sample.

### B. Results

### 1. Environmental Stress Cracking

TABLE 7
The composition of formulations 1 according to the invention and a comparative
formulation 2, which were used for the environmental stress cracking test.

| | | | | 2- | | | Decyl | Poly |
|---|---|---|---|---|---|---|---|---|
| Form- | | | Phenoxy | Phenyl | | Synperonic | amine | quaternium |
| ulation | BZK | DDAC | ethanol | phenol | 91-6 | PEL64 | oxide | 6 |
| 1 | 0.27 | 0.27 | 0.3 | 0 | 0.06 | 0 | 0.15 | 0.05 |
| 2 | 0.54 | 0.54 | 0.6 | 0.1 | 0.99 | 0.12 | 0 | 0 |
| | | | | | | | | Allergen |
| | | | | | | | | Free |
| | | | | | Di- | | | Green |
| | Form- | | Propylene | | sodium | Citric | Sodium | Tea |
| | ulation | Glycerol | Glycol | Allantoin | EDTA | acid | Citrate | Perfume |
| | 1 | 0 | 0 | 0 | 0.03 | 0 | 0 | 0.0075 |
| | 2 | 0 | 0.2 | 0 | 0 | 0.01 | 0.1 | 0.05 |

TABLE 8
Results of ESC testing of five polymer specimens for each of the two formulations at 0.5% strain.
For specimens, which were seen to crack, the time following initial exposure is noted.

| Formulation | Formulations 1 | Formulations 2 |
|---|---|---|
| Active Ingredients | Benzalkonium chloride - 0.27% | Benzalkonium chloride - 0.54% |
| | Didecyl dimethylammonium chloride - 0.27% | Didecyl dimethylammonium chloride - 0.54% |
| pH | 5.8 | 5.5 |
| Polycarbonate, Medium | Pass - no cracking or crazing, no significant change in | Tensile Failure - tensile properties weakened |
| Viscosity | tensile properties | |
| Polycarbonate, High | Pass - no cracking or crazing, no significant change in | Pass - no cracking or crazing, no significant |
| Viscosity | tensile properties | change in tensile properties |
| Polycarbonate and ABS | Pass - no cracking or crazing, no significant change in | Tensile Failure - tensile properties weakened |
| blend | tensile properties | |
| ABS | Pass - no cracking or crazing, no significant change in | Failure - cracking observed after 120 hours of |
| | tensile properties | exposure |
| PMMA | Pass - no cracking or crazing, no significant change in | Failure - cracking observed after 12 hours of |
| | tensile properties | exposure |

TABLE 9
Results of ESC testing of five polymer specimens for each of the two formulations 1 and 2 and three competitor formulations
A, B, and C at 0.5% strain. For specimens, which were seen to crack, the time following initial exposure is noted.

| | Formulation | | | | |
|---|---|---|---|---|---|
| Product | 1 | 2 | Competitor A | Competitor B | Competitor C |
| Active | Benzalkonium chloride - | Benzalkonium | Didecyl | Didecyl | Didecyl |
| Ingredients | 0.27% | chloride - 0.54% | dimethylammonium | dimethylammonium | dimethylammonium |
| | Didecyl | Didecyl | chloride - 0.64% | chloride - 0.45% | chloride - 0.44% |
| | dimethylammonium | dimethylammonium | Dodecyl dipropylene | Ethanolamine < 0.6% | Na2CO3 < 2% |
| | chloride - 0.27% | chloride - 0.54% | triamine - 0.53% | | Ethylene glycol < 1% |
| | | | Ethanolamine < 1% | | |
| | | | Ethoxylated | | |
| | | | Alcohol < 1% | | |
| pH | 5.8 | 5.5 | 10.5 | 10.8 | 8.8 |
| Polycarbonate | Pass - no cracking or | Tensile Failure - | Failure - cracking | Failure - cracking | Failure - cracking |
| (Medium | crazing, no significant | tensile properties | observed after 12 | observed after 36 | observed after 36 |
| Viscosity) | change in tensile | weakened | hours of exposure | hours of exposure | hours of exposure |
| | properties | | | | |
| Polycarbonate | Pass - no cracking or | Pass - no cracking | Failure - cracking | Failure - cracking | Failure - cracking |
| (High | crazing, no significant | or crazing, no | observed after 36 | observed after 36 | observed after 36 |
| Viscosity) | change in tensile | significant change in | hours of exposure | hours of exposure | hours of exposure |
| | properties | tensile properties | | | |
| Polycarbonate | Pass - no cracking or | Tensile Failure - | Failure - cracking | Pass - no cracking or | Failure - cracking |
| and ABS blend | crazing, no significant | tensile properties | observed after 48 | crazing, no significant | observed after 48 |
| | change in tensile | weakened | hours of exposure | change in tensile | hours of exposure |
| | properties | | | properties | |
| ABS | Pass - no cracking or | Failure - cracking | Pass - no cracking or | Pass - no cracking or | Tensile Failure - |
| | crazing, no significant | observed after 120 | crazing, no significant | crazing, no significant | tensile properties |
| | change in tensile | hours of exposure | change in tensile | change in tensile | weakened |
| | properties | | properties | properties | |
| PMMA | Pass - no cracking or | Failure - cracking | Failure - cracking | Pass - no cracking or | Failure - cracking |
| | crazing, no significant | observed after 12 | observed after 120 | crazing, no significant | observed after 24 |
| | change in tensile | hours of exposure | hours of exposure | change in tensile | hours of exposure |
| | properties | | | properties | |

### C. Comments

7 days exposure at the ambient conditions of 23° C./50% RH was used as test conditions owing to long lifetimes of devices using the tested plastic. In use, Clinell wipes wet a surface for ˜2 minutes per wipe, 7 days of exposure therefore represents a significant number of wipes.

For the tested polymer at the 0.5% strain level, comparative formulation 2 and the competitor formulations A, B, and C are deemed to be incompatible due to ESC failure at this exposure period. In contrast, the composition according to the invention showed superior compatibility with no significant ESC effect for the same polymer at the same strain level.

1. A composition comprising at least one quaternary ammonium compound, at least one alcohol ethoxylate, at least one chelating agent, and at least one amine oxide, wherein the at least one quaternary ammonium compound comprises at least one polyquaternium compound.

2. The composition according to claim 1, wherein the at least one polyquaternium compound is selected from polyquaternium-1, polyquaternium-2, polyquaternium-4, polyquaternium-5, polyquaternium-6, polyquaternium-7, polyquaternium-8, polyquaternium-9, polyquaternium-10, polyquaternium-11, polyquaternium-12, polyquaternium-13, polyquaternium-14, polyquaternium-15, polyquaternium-16, polyquaternium-17, polyquaternium-18, polyquaternium-19, polyquaternium-20, polyquaternium-22, polyquaternium-24, polyquaternium-27, polyquaternium-28, polyquaternium-29, polyquaternium-30, polyquaternium-31, polyquaternium-32, polyquaternium-33, polyquaternium-34, polyquaternium-35, polyquaternium-36, polyquaternium-37, polyquaternium-39, polyquaternium-42, polyquaternium-43, polyquaternium-44, polyquaternium-45, polyquaternium-46, polyquaternium-47, and any mixtures thereof.

3. The composition according to claim 1, wherein the at least one polyquaternium compound is polyquaternium-6 (poly(dialyl)dimethylammoniumchloride), polyquaternium-4 (Hydroxyethyl cellulose dimethyl diallylammonium chloride copolymer), polyquaternium-5 (Copolymer of acrylamide and quaternized dimethylammoniumethyl methacrylate), polyquaternium-7 (Copolymer of acrylamide and diallyldimethylammonium chloride), polyquaternium-10 (Quaternized hydroxyethyl cellulose), polyquaternium-11 (Copolymer of vinylpyrrolidone and quaternized dimethylaminoethyl methacrylate), polyquaternium-14 (Trimethylaminoethylmethacrylate homopolymer), polyquaternium-16 (Copolymer of vinylpyrrolidone and quaternized vinylimidazole), polyquaternium-22 (Copolymer of acrylic acid and diallyldimethylammonium Chloride), polyquaternium-28 (Copolymer of vinylpyrrolidone and methacrylamidopropyl trimethylammonium), polyquaternium-29 (Chitosan modified with propylene oxide and quaternized with epichlorhydrin), polyquaternium-39 (Terpolymer of acrylic acid, acrylamide and diallyldimethylammonium Chloride), polyquaternium-46 (Terpolymer of vinylcaprolactam, vinylpyrrolidone, and quaternized vinylimidazole), or any mixtures thereof.

4. The composition according to claim 1, wherein the at least one alcohol ethoxylate comprises alcohol ethoxylate nonionic with HLB from 4-16.

5. The composition according to claim 1, wherein the at least one alcohol ethoxylate comprises alcohol ethoxylate nonionic with HLB from 8-14, preferably 11-13.

6. The composition according to claim 1, wherein the at least one alcohol ethoxylate comprises alcohol ethoxylate nonionic C9-11, EO6 (C9-11 pareth 6).

7. The composition according to claim 1, wherein the at least one chelating agent comprises Disodium ethylenediaminetetraacetic acid; and/or
wherein the at least one amine oxide comprises N,N-dimethyldecylamine-N-oxide; and/or
wherein the at least one quaternary ammonium compound additional comprises benzalkonium chloride and/or didecyldimethylammonium chloride; and/or
wherein the at least one polyquaternium compound is present in an amount of 0.01 to 1.0% w/w; and/or
wherein the composition comprises a further quaternary ammonium compound in form of C12-C16 Benzalkonium Chloride, in particular in an amount of 0.01 to 0.7% w/w; and/or
wherein the composition comprises a further quaternary ammonium compound in form of Didecylydimethyl ammonium chloride, in particular in an amount of 0.01 to 0.7% w/w; and/or
wherein the at least one alcohol ethoxylate is present in an amount of 0.01 to 0.1% w/w; and/or
wherein the at least one amine oxide is present in an amount of 0.01 to 1% w/w.

8-14. (canceled)

15. The composition according to claim 1, wherein the at least one chelating agent is present in an amount of 0.01 to 1% w/w.

16. The composition according to claim 1, wherein the composition further comprises one or more additives selected from a group consisting of one or more disinfectants, stabilizers, preservatives, dyes, fragrances, odor masking agents and/or mixtures thereof.

17. The composition according to claim 1, in the form of an aqueous or aqueous alcohol solution or dispersion.

18. The composition according to claim 1, wherein the composition comprises:
from 0.01 to 0.25% w/w N,N-dimethyldecylamine-N-oxide,
from 0.01 to 0.1% w/w Alcohol exthoxylate nonionic C9-11, E06,
from 0.01 to 1.0% w/w Poly(dialyl dimethylammoniumm Chloride),
from 0.01 to 1.0% w/w Disodium ethylenediaminetetraacetic acid,
with the solvent being water.

19. The composition according to claim 1, wherein the composition comprises:
from 0.01 to 0.7% w/w C12-C16 Benzalkonium Chloride,
from 0.01 to 0.7% w/w Didecylydimethyl ammonium chloride,
from 0.01 to 0.25% w/w N,N-dimethyldecylamine-N-oxide,
from 0.01 to 0.1% w/w Alcohol Exthoxylate nonionic C9-11, E06,
from 0.01 to 1.0% w/w Poly(dialyl dimethylammoniumm Chloride),
from 0.01 to 1.0% w/w Disodium ethylenediaminetetraacetic acid,
with the solvent being water.

20. The composition according to claim 1, wherein the composition comprises:
from 0.01 to 1.0% w/w Phenoxyethanol, such as 0.01 to 0.5% w/w,
from 0.01 to 0.7% w/w C12-C16 Benzalkonium Chloride,
from 0.01 to 0.7% w/w Didecylydimethyl ammonium chloride,
from 0.01 to 0.25% w/w N,N-dimethyldecylamine-N-oxide,
from 0.01 to 0.1% w/w Alcohol Exthoxylate nonionic C9-11, E06,
from 0.01 to 1.0% w/w Poly(dialyl dimethylammoniumm Chloride),
from 0.01 to 1.0% w/w Disodium ethylenediaminetetraacetic acid,
with the solvent being water.

21. The composition according to claim 1, which is free of polyhexamethylene biguanide (PHMB) and phenols.

22. The composition according to claim 1, which is free of 2-phenoxy ethanol.

23. The composition according to claim 1, wherein the composition is in the form of an aqueous solution having a pH of from 3 to 9, preferably from 4 to 8, more preferably from 5 to 7.

24. A wet wipe comprising a substrate that has been impregnated with the composition according claim 1.

25. The wet wipe according to claim 24, wherein the substrate comprises fibres made of any of or a mixture of polypropylene, polyester, polyethylene, viscose, cotton, regenerated wood pulp, cellulose, micro-fibres and nano-fibres.

26. The wet wipe according to claim 24, wherein the substrate being packaged ready to be dispensed from a tub, a bucket, a flow-wrap pack or an individually sealed wrapper.

27. A method comprising:
using a composition according to claim 1 or a wet wipe that comprises said composition for disinfection of a surface.
